(19) United States
(12) Patent Application Publication
(10) Pub. No.: US 2010/0221769 A1
(43) Pub. Date: Sep. 2, 2010

(54) ELECTROPORATIVE FLOW CYTOMETRY

(76) Inventors: Chang LU, West Lafayette, IN (US); Jun Wang, West Lafayette, IN (US); Ning Bao, West Lafayette, IN (US); Robert L. Geahlen, West Lafayette, IN (US)

Correspondence Address: BRINKS HOFER GILSON & LIONE, P.O. BOX 10395, CHICAGO, IL, 60610, US

(21) Appl. No.: 12/716,974

(22) Filed: Mar. 3, 2010

Related U.S. Application Data
(63) Continuation of application No. PCT/US2008/075088, filed on Sep. 3, 2008.
(60) Provisional application No. 60/967,407, filed on Sep. 4, 2007.

(51) Int. Cl. C12Q 1/02; C12M 1/34

(52) U.S. Cl. 435/29; 435/288.7

(57) ABSTRACT

Novel devices and methods are provided, which include electroporative flow cytometry, where electroporation is combined with flow cytometry. The devices and methods can be used for the detection a variety of cellular features, including protein translocation, and for monitoring biomechanics at single cell level. Using the novel devices methods it is possible to observe the release of proteins such as intracellular kinases out of the cells during electroporation. Using the novel devices methods it is possible to study cytoskeleton dynamics and deformability at a single cell level, and to correlate these to diseases such as cancers.

### CROSS-REFERENCE TO RELATED APPLICATIONS

This invention claims priority to U.S. Provisional Patent Application Ser. No. 60/967,407, filed Sep. 4, 2007, which is herein incorporated by reference.

### GOVERNMENT INTERESTS

This invention was made with United States government support from the National Cancer Institute, grant number CA37372, and by the National Science Foundation, grant number CBET-0747105. The United States government has certain rights in this invention.

### FIELD OF THE INVENTION

This invention relates to the fields of fluidic devices, electroporation, flow cytometry, protein translocation, and cell biomechanics.

### BACKGROUND

Translocation of a protein between different subcellular compartments is a common event during signal transduction in living cells. Integrated signaling cascades often lead to the relocalization of protein constituents such as translocations between the cytoplasm and the plasma membrane or nucleus. Such events can be essential for the activation/deactivation and biological function of the protein.

The protein-tyrosine kinase, Syk, is a prime example of a protein that translocates to the plasma membrane as part of its role in signal transduction. Syk is essential for the survival, proliferation and differentiation of B lymphocytes, processes regulated by signals sent from the cell surface receptor for antigen, BCR (Takata et al., 1994, EMBO J. 13: 1341-1349; Turner et al., 1995, Nature 378: 298-302). Syk is the prototype kinase of the Syk/Zap-70 family (Zioncheck et al., 1988, J. Biol. Chem. 263: 19195-19202). In mature B cells, the BCR comprises a polymorphic, membrane-associated immunoglobulin that bears the antigen combining site in association with a disulfide-linked heterodimer of CD79a and CD79b. Clustering of the BCR by interactions with antigens (or artificially through interactions with anti-IgM antibodies) leads to the phosphorylation of a pair of tyrosines located within immunoreceptor tyrosine-based activation motifs (ITAMs) located on the cytoplasmic tails of CD79a and CD79b. This leads to the recruitment to the receptor of Syk, which binds to the phosphorylated receptor through a tandem pair of N-terminal SH2 domains and couples the receptor to multiple intracellular signaling networks including the Ras/Erk, phospholipase Cγ/NF-AT and PI3K/Akt pathways (DeFranco, 1997, Curr. Opin. Immunol. 9: 296-308; Campbell, 1999, Curr. Opin. Immunol. 11: 256-264).

Determination of the translocation of proteins within cells has been traditionally carried out using methods such as subcellular fractionation/Western blotting or imaging of a few cells. However, when heterogeneous cell populations (e.g., samples derived from primary materials) are studied, it is always beneficial to obtain quantitative information of the entire population at the single cell level to prevent cell subsets from being overlooked. Flow cytometry has been the tool of choice for single cell studies within cell populations of relatively large sizes. However, conventional flow cytometry is intrinsically insensitive to the subcellular location of the probed protein. Laser scanning cytometry (LSC) quantifies nuclear/cytoplasmic distribution of a fluorescently labeled protein based on solid phase cytometry technique (Deptala et al., 1998, Cytometry 33: 376-382; Bedner et al., 2000, Cytometry 41: 83-88; Ozawa et al., 2005, Cytometry Part A 65A: 69-76). However, the algorithm of quantification based on image analysis is complex and lacks robustness—the throughput is typically less than 100 cells/second, compared to about 10⁴ cells/second for flow cytometry (Pozarowski et al., 2005, In: Cell Imaging Techniques: Methods and Protocols, Taatjes and Mossman, eds., Humana Press, Totowa, N.J., Vol. 319, pp 165-192). Flow cytometric screening of isolated nuclei has also been applied to the study of nucleus/cytoplasm translocation (Blaecke et al., 2002, Cytometry 48: 71-79; Cognase et al., 2003, Immunol. Lett. 90: 49-52). This technique requires additional steps to isolate nuclei from living cells and the sample may potentially be contaminated by other organelles. A technique based on the manipulation of molecular biology inside cells, complementation assay for protein translocation (CAPT), was recently developed (Wehrman et al., 2005, Nat. Methods 2: 521-527), which, combined with flow cytometry, was able to analyze intracellular translocation from the cytoplasm to the nucleus or plasma membrane. Although interesting for applications to mechanistic studies, the rather complex molecular biology makes this technique impractical for clinical applications.

Biomechanical properties of cells have important implications for cell signaling, cytoadherence, migration, invasion and metastatic potential. Mechanical properties of cells such as deformability largely depend on physical properties of the cytoskeleton, the internal scaffolding comprising a complex network of biopolymeric molecules. During the development of diseases such as cancers, the structures of the cytoskeleton and the extracellular matrix are often transformed. During the cell's progression from a fully mature, postmitotic state to an immortal cancerous cell, the cytoskeleton experiences a reduction in the amount of constituent polymers and accessory proteins and a restructuring of the biopolymeric network. The altered cytoskeleton changes the ability for cancer cells to contract or stretch. As a result, malignant cells exhibit lower resistance to deformation than normal cells and metastatic cancer cells are even more deformable than nonmetastatic cells.

Over the years there have been a number of tools developed for measuring biomechanical properties of cells and establishing their relationships to malignancy and metastasis. Early techniques including filtration and micropipette aspiration were used to study the deformation properties of nontransformed and transformed cells. It was found that a direct correlation exists between an increase in deformability and progression from a nontumorigenic cell line to tumorigenic and metastatic cell line (Ochalek et al., 1988, Cancer Res. 48: 5124-5128; Ward et al., 1991, Biorheology 28: 301-313). Atomic force microscopy (AFM) has also been used to probe an attached cell by applying a local force and measuring the deformation and local structural properties using a hard indentor. AFM data revealed that normal cells have a Young's modulus of about one order of magnitude higher than cancerous ones (Lekka et al., 1999, Eur. Biophys. J. 28: 312-316). AFM was also recently applied to nanomechanical studies of cells from cancer patients (Cross et al., 2007, Nat. Nanotechnol. 2: 780-783). Magnetic tweezers have been applied to study the viscoelastic properties of cells by attaching magnetic beads on cell surface and exerting magnetic forces while tracking the bead locations. Similarly, optical tweezers have been applied to study cell elasticity and mechanotransduction by manipulating beads attached to cell surface. In microplate manipulation, a cell can be seized between two microplates with the more flexible one serving as a sensor of the applied force while unidirectional compression and traction is applied. Microfluidic channels with cross-sectional area smaller than that of cells were applied and the behavior of cells squeezing through was observed for characterizing cell deformability (Shelby et al., 2003, Proc. Natl. Acad. Sci. USA 100: 14618-14622; Suresh, 2007, Acta Biomater. 3: 413-438).

In spite of the variety of the techniques demonstrated, most of the above techniques are only capable of studying a low number of single cells due to their low throughput. The data generated by these techniques are not necessarily representative of a large cell population. This problem becomes particularly serious when a heterogeneous cell population consisting of different cell types (e.g. tissue samples derived from animals and patients) is involved. Recent effort has been directed to improve the throughput of cell biomechanical assays. A microfluidic optical stretcher was developed to examine the elasticity of cells in a continuous flow at a throughput of 1 cell/min (Guck et al., 2005; Biophys. J. 88: 3689-3698). In this experiment, the flowing cells were captured and deformed in a two-beam laser trap by optical forces. Although greatly improved compared to earlier tools, the throughput of 1 cell/min is still orders of magnitude lower than that of high throughput single cell techniques such as flow cytometry (about 10⁴ cells/s). The low throughput issue may hinder the wide application of biomechanical assays as effective tools for cancer diagnosis and staging.

Electroporation occurs when cells experience an external electrical field with the intensity beyond a certain threshold. During electroporation the electrical field opens up pores in the cell membrane which allow material exchange across the membrane. Swelling, or an expansion in the cell size, is a well-known phenomenon associated with electroporation (Ferret et al., 2000, Biotechnol. Bioeng. 67: 520-528; Deng et al., 2003, Biophys. J. 84: 2709-2714). Swelling is due to influx of water and small molecules into cells when the membrane is breached by electroporation. Such influx is in general believed to be a result of osmotic pressure imbalance inside and outside the cells (Tsong, 1991, Biophys. J. 60: 297-306; Wang and Lu, 2006, Biotechnol. Bioeng. 95: 1116-1125). Although related, such swelling during electroporation is mechanistically different from electrodeformation (Wong et al., 2005, J. Biomech. 38: 529-535). The latter refers to the deformation of cells by dielectric forces with polarity due to the application of electric field. Electrodeformation does not necessarily require poration of the membrane.

Therefore, simple and robust high-throughput methods for examining intracellular protein translocation and biomechanical properties of cells at the single cell level are highly desired for mechanistic studies and clinical applications. The invention described herein addresses these and related needs.

### SUMMARY OF THE INVENTION

Provided is a novel tool, referred to as electroporative flow cytometry, to detect the translocation of cellular proteins. Electroporation occurs when cells experience an electrical field with the intensity beyond a certain threshold. During electroporation the electrical field opens up pores in the cell membrane. Such pores allow the release of intracellular materials into the surrounding solution (Rols and Teissie, 1990, Biophys. J. 58: 1089-1098; Wang and Lu, 2006, Chem. Commun. 3528-3530; Wang and Lu, 2006, Anal. Chem. 78: 5158-5164). It is possible to differentiate a cell population with translocation from one without it with the information collected from individual cells of the entire population. This technique allows detection of protein translocation at the single cell level. Due to the frequent involvement of kinase translocations in disease processes such as oncogenesis, the methods of the present invention have utility in kinase-related drug discovery and tumor diagnosis and staging.

In one example, the devices and methods of the present invention can be used to detect the intracellular translocation of the kinase Syk from the cytoplasm to plasma membrane at the level of the cell population with information gathered from single cells. In one example, cells are flowed through a microfluidic channel where each cell experiences a high electroporation field in a predefined section for the same duration and then is detected based on laser-induced fluorescence under hydrodynamic focusing. The amount of followed Syk left in the cells after electroporation is related to whether or not translocation of Syk to the plasma membrane occurs. A cell population stimulated with anti-IgM (which induces translation of Syk to the plasma membrane) had more Syk remaining in cells after the electroporation than the population that is not stimulated. Thus, EFC is able to detect the translocation of an intracellular kinase Syk and provide characteristics of the entire cell population in terms of the release of the kinase.

The methods of the present invention can be extended to the detection of translocations involving other kinases and cell types.

Electroporative flow cytometry can also be applied to biomechanics of the cell and the cellular components. For example, ECF can be used to detect one or more cells with deformed cytoskeleton, and to detect and identify one or more diseased cells. In particular, the methods can be applied to detect and identify cells where deformations of the cytoskeleton are correlated with cell membrane permeability and/or changes in the cell size.

Devices and methods are provided, which include: (a) subjecting a population of cells to electroporation; (b) subjecting the population of electroporated cells to flow cytometry; and (c) detecting flow cytometry-related data associated with a characteristic cellular feature.

In the practice of the methods, the detection of cellular features may include detection of one or more of morphological, phenotypical, and/or physiological features of the examined cells and the cellular components. The detection of cellular features can occur at one or multiple points before, during and/or after electroporation. Electroporation can be carried out in a variety of ways, for example, using conventional pulse-based electroporation, using flow-through electroporation, or combinations thereof.

### BRIEF DESCRIPTION OF THE DRAWINGS

FIG. 1 schematically illustrates the layout of the device fabricated on a microfluidic chip (a); and the setup of the microfluidic electroporative flow cytometry apparatus (b).

FIG. 2 shows fluorescent images showing the translocation of SykEGFP to the plasma membrane. SykEGFP was redistributed to the plasma membrane after stimulation with anti-IgM antibody at room temperature for 5 min (a), 30 min (b), 60 min (c), and 90 min (d).

FIG. 3 shows graphs of histograms of the fluorescent intensity of SykEGFP-DT40-Syk⁻-Lyn⁻ cells with and without stimulation by anti-IgM antibody and the DT40-Syk⁻-Lyn⁻ cells (control): (a) histograms obtained by Cytomics FC 500 Flow cytometer; (a) Analysis of the same samples as in (a) in the microfluidic device (with the voltage at 0).

FIG. 4 shows histograms of the fluorescent intensity of DT40 cells detected by the microfluidic EFC under different electric field intensities and durations. SykEGFP-DT40-Syk⁻-Lyn⁻ cells were applied in (a) and (b), while calcein AM stained DT40-Syk⁻-Lyn⁻ cells were used in (c). The black curves show data from cells stimulated with anti-IgM and the gray curves show data from cells that were not stimulated.

FIG. 5 shows graphs illustrating the variation of the mean fluorescence intensity value of the cell population at different field intensities with and without stimulation by anti-IgM. SykEGFP-DT40-Syk⁻-Lyn⁻ cells were applied in (a) and (b), while calcein AM stained DT40-Syk⁻-Lyn⁻ cells were used in (c).

FIG. 6 shows graphs illustrating the quantitative analysis of the fluorescence intensity of DT40 cells: (a) Histogram of the fluorescence intensity from a mixture of beads with varying predefined fluorescence intensity levels; (b) Calibration curve with the MESF values of the beads plotted against the peak channel numbers (the fluorescent intensities) of the beads obtained by the microfluidic system (R²=0.9978, solid square); (c) Relative EGFP content in SykEGFP-DT40-Syk⁻-Lyn⁻ cells under different electric field intensities with electroporation duration of 120 ms; (d) Relative EGFP content in SykEGFP-DT40-Syk⁻-Lyn⁻ cells under different electric field intensities with electroporation duration of 60 ms; (e) Relative calcein content in calcein AM stained DT40-Syk⁻-Lyn⁻ under different electric field intensities with a electroporation duration of 60 ms.

FIG. 7 shows images of SykEGFP-DT40-Syk⁻-Lyn⁻ cells after the microfluidic EFC screening with different field intensities and field duration of 60 ms.

FIG. 8 shows images of SykEGFP-DT40-Syk⁻-Lyn⁻ cells after the microfluidic EFC screening with different field intensities and field duration of 120 ms.

FIG. 9 shows a graph and an image of Western blotting analysis of the SykEGFP fraction in the cytosol and on the membrane with and without stimulation by anti-IgM antibody.

FIG. 10 is a schematic illustration of another embodiment of the microfluidic electroporative flow cytometry setup. The inset image shows the cell size change of the same cell (MCF-7) at different time points and at different locations while it is flowing in the channel with the field intensity of 400 V/cm in the narrow section.

FIG. 11 shows graphs illustrating the variation in the cell size during flow-through electroporation for MCF-10A, MCF-7 and TPA-treated MCF-7 cells, under field intensities in the narrow section of 600 V/cm (A), 400 V/cm (B), and 200 V/cm (C).

FIG. 12 shows images illustrating the morphological change of the cell during flow-through electroporation and the release of intracellular materials. A TPA-treated MCF-7 cell was monitored at different time points: (A) 0 ms; (B) 64 ms; (C) 128 ms; (D) 192 ms; and (E) 256 ms and at various locations in the narrow section.

FIG. 13 is a graph showing histograms of the swelling of MCF-10A, MCF-7 and TPA-treated MCF-7 cells under the field intensity of 400 V/cm (in the narrow section) and at the time point of 192 ms.

FIG. 14 is a graph showing histograms of the swelling of MCF-7 and colchicine-treated MCF-7 cells under the field intensity of 600 V/cm (in the narrow section) and at the time point of 192 ms.

FIG. 15 is a graph showing the viability of MCF-10A, MCF-7 and TPA-treated MCF-7 cells after flow-through electroporation of 200 ms with various field intensities in the narrow section.

### DETAILED DESCRIPTION OF THE PREFERRED EMBODIMENTS

Unless defined otherwise, all technical and scientific terms used herein have the meaning commonly understood by a person skilled in the art to which this invention belongs. The following references provide one of skill with a general definition of many of the terms used in this invention: Singleton et al., 1994, Dictionary Of Microbiology And Molecular Biology, 2nd ed., John Wiley and Sons, NY; The Cambridge Dictionary Of Science And Technology, 1988, Walker ed., Cambridge University Press, Cambridge, UK; The Glossary Of Genetics, 1991, 5^{th} ed., Rieger et al., eds., Springer Verlag, Berlin, Germany; and Hale and Markham, 1991, The Harper Collins Dictionary Of Biology, Harper Perennial, NY. As used herein, the following terms have the meanings ascribed to them unless specified otherwise.

A “flow channel” refers generally to a flow path through which a solution can flow.

“Flow cytometry” is a technique for counting, examining, and/or sorting small particles (for example, cells) suspended in a stream of fluid. It allows simultaneous multiparametric analysis of the physical and/or chemical characteristics of single cells flowing through an optical and/or electronic detection apparatus. In flow cytometry, a beam of light (usually laser light) of a single wavelength is directed onto a hydro-dynamically focused stream of fluid. Typically, a number of detectors are aimed at the point where the stream passes through the light beam; one in line with the light beam (Forward Scatter or FSC) and one or more perpendicular to it (Side Scatter (SSC). There may be one or more fluorescent detectors. Each suspended particle (e.g. cell) passing through the beam scatters the light in some way, and compounds (e.g. fluorescent chemicals) found in the particle or attached to the particle may be excited into emitting light at a higher wavelength than the light source. This combination of scattered and fluorescent light is picked up by the detectors, and by analyzing fluctuations in brightness at each detector (one for each fluorescent emission peak) it is then possible to derive various types of information about the physical and chemical structure of each individual particle. FSC correlates with the cell volume and SSC depends on the inner complexity of the particle (e.g. shape of the cytoskeleton). Some flow cytometers on the market have eliminated the need for fluorescence and use only light scatter for measurement. Other flow cytometers form images of each cell's fluorescence, scattered light, and transmitted light. All of the above variations of flow cytometry are contemplated in the practice of the present invention.

The term “constant direct current voltage” refers to the voltage of constant magnitude over time, which is typically generated by a direct current power supply.

“Electroporation” or “electropermeabilization” refers to a significant increase in the electrical conductivity and permeability of the cell plasma membrane caused by an externally applied electric field.

“Flow through electroporation” refers to electroporation that is based on the application of constant DC voltage while varying the size and/or dimensions of fluidic channels in different sections, for example as described in Wang and Lu, 2006, Anal. Chem. 78: 5158-5164; and in U.S. Patent Application Pub. No. US 2007/0105206 A1, both of which are herein incorporated by reference.

“Electroporative flow cytometry” refers to a method that combines various embodiments of electroporation with various embodiments of flow cytometry. As described herein, flow-through electroporation in a microfluidic channel with geometric variation provides an ideal and high-throughput platform for combining flow cytometry with electroporation.

The phrase “electric field intensity threshold for electroporation” refers to the strength of an electric field that will cause pores to form in the plasma membrane. Typically this occurs when the voltage across a plasma membrane exceeds its dielectric strength. If the strength of the applied electric field and/or duration of exposure to it are properly chosen, the pores formed by the electrical pulse reseal after a short period of time, during which extracellular compounds have a chance to enter into the cell. However, excessive exposure of live cells to electric fields can cause apoptosis and/or necrosis—the processes that result in cell death.

Electroporation is usually used in molecular biology as a way of introducing some substance into a cell, such as loading it with a molecular probe, a drug that can change the cell's function, or a piece of coding DNA. Electroporation with increased strength and/or duration of the electric field can lead to cell lysis and release of cellular materials. Aspects of the electroporation technique may be based on the techniques disclosed in U.S. Patent Application Pub. No. US 2007/0105206 A1 (“Fluidic device”); U.S. patent Application Pub. No. US 2006/0269531 A1 (“Apparatus for generating electrical pulses and methods of using the same”); and U.S. patent Application Pub. No. US 2006/0062074 A1 (“Method for intracellular modifications within living cells using pulsed electric fields”); and in U.S. Pat. No. 5,128,257 (“Electroporation apparatus and process”), all of which are herein incorporated by reference.

Aspects of flow cytometry useful in the practice of the present invention are disclosed in U.S. Pat. Nos. 4,673,288 and 4,818,103 (“Flow cytometry”); U.S. Pat. No. 4,786,165 (“Flow cytometry and apparatus therefore”); U.S. Pat. No. 4,660,971 (“Optical features of flow cytometry apparatus”); and in U.S. Pat. No. 4,661,913 (“Apparatus and method for the detection and classification of articles using flow cytometry techniques”), all of which are herein incorporated by reference.

“Biomechanics” is the application of mechanical principles on living organisms. Biomechanics, as used herein, in particular refers to the loads and deformations that can affect the features of cells, and more particularly it refers to the features of the cellular cytoskeleton.

“Swelling” is the enlargement of cells caused by accumulation of excess fluid in the cells. The relative amount of cell swelling can be expressed through a relative increase in cell size. “Cell size”, as used herein, refers to the volume of a cell and how much three-dimensional space it occupies, and can be quantified numerically.

“Deformability”, as used herein, refers to a difference in the shape of a cell compared to the average shape for the cell in question. Deformability may arise from numerous causes. In particular, cell deformability may arise from any type of deformation in the underlying cell cytoskeleton (microtubules, actin, intermediate filaments, etc.). Importantly, as described herein, cell deformation and changes in the mechanical properties of cytoskeleton may be associated with disease, and in particular with malignant diseases, i.e. the ability of the cells to contract/expand may change because a disease may influence the composition of the cytoskeleton. The particular type of cytoskeleton deformation is not important for the practice of the present invention. What is important is that the alteration, i.e. change in the cytoskeleton and/or plasma membrane allows for increased cell swelling, i.e. increased cell size, when the cell with altered cytoskeleton is treated according to the methods of the present invention.

Detection of cell size can be performed in a variety of ways. Preferably, detection of the cell size comprises measuring the two-dimensional area of individual cells in time-sequenced images. In some example, this can be done using various software packages, e.g. ImageJ software from the National Institutes of Health (NIH).

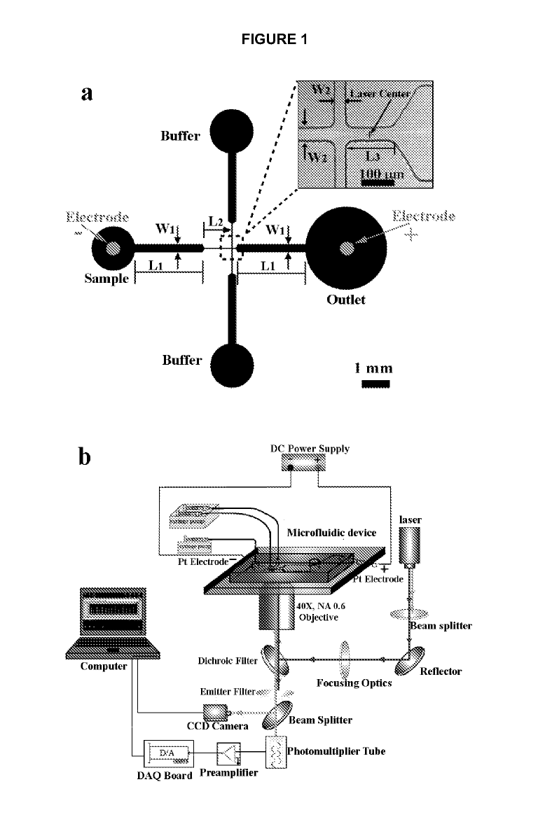

A new system that includes an electroporation technique in combination with flow cytometry is disclosed herein. Some aspects of the present invention are disclosed in Wang and Lu, 2006, Biotechnol. Bioeng. 95: 1116-1125; and in Wang and Lu, 2006, Anal. Chem. 78: 5158-5164, both of which are incorporated herein by reference. A horizontal channel may include two wide sections (W₁=300 μm) and one narrow section (W₂=30 μm) with the depth of the whole channel being uniform (FIG. 1a). The electric field strength (electric field intensity) in the narrow section is higher than the electric field strength (electric field intensity) in the wide sections. Embodiments include devices where the electric field strength in the narrow section is approximated to be 10 times higher than the electric field strength in the wide sections according to Ohm's law, which predicts that the electric field strength in each section is inversely proportional to the cross-sectional area in the section under a constant DC voltage. A similar field intensity distribution in the channel was modeled in the inventors' previous work (Wang and Lu, 2006, Appl. Phys. Lett. 89: 234102), which is herein incorporated by reference.

When the overall voltage is in the right range, electroporation exclusively occurs in the narrow section since the field intensity in the wide sections is too weak to compromise the cell membrane (Wang and Lu, 2006, Biotechnol. Bioeng. 95: 1116-1125; Wang and Lu, 2006, Anal. Chem. 78: 5158-5164). In such a flow-through electroporation device, the duration for cells to stay in the narrow section (the electroporation section) of the channel can be determined (and adjusted) by their velocity and the length of the section. The velocity of cells can, for example, be determined by the infusion rate of an accompanying syringe pump. The electrical field has little effect on the velocity. A detector can also be used. For example, a light detection point can be positioned in the narrow section after the point where hydrodynamic focusing occurs (FIG. 1a). This special constant-voltage based electroporation technique provides a unique design for treating single cells uniformly when a stable flow was established. The microfluidic EFC device can be fabricated using a variety of substrates, for example on a PDMS/glass slide using standard soft lithography as previously demonstrated (Wang and Lu, 2006, Anal. Chem. 78: 5158-5164).

Shown in FIG. 1 is one embodiment of the devices and methods of the present invention. In preferred embodiments, this device can particularly be used for the detection of protein translocation at the single cell level. FIG. 1(a) is a layout of the device that is fabricated on a microfluidic chip. The width varied in the horizontal channel may vary. Preferably the width W₁ is about 300 μm and the width W₂ is about 33 μm. The length of the narrow section L₂ is preferably set at about 1-2 mm. The other sections in the horizontal channel can have various lengths, for example L₁ has a preferred length of about 2.5 mm, and L₃ has a preferred length of about 150 μm. The depth of the microfluidic channels may vary, and it is preferably uniform and about 33 μm. A laser detection point is positioned, preferably at the center of the horizontal channel, after hydrodynamic focusing. In one example, shown in the inset image in FIG. 1, the fluorescent trail was left by SykEGFP-DT40-Syk⁻-Lyn⁻ cells when the ratio between the flow rate in one of the two vertical channels and that in the horizontal channel was 3:1. FIG. 1(b) is a schematic illustration of the setup of the microfluidic electroporative flow cytometry apparatus.

In one preferred embodiment, the design and setup of the microfluidic EFC device includes two intersecting microfluidic channels connected to four reservoirs (FIG. 1a). However, the number of channels and reservoirs may vary. The dimensions of the channels and reservoirs also may vary. Preferably, the cell sample flows through the horizontal channel from the sample reservoir to the outlet reservoir, carried by a pressure-driven flow generated by a syringe pump (FIG. 1b). In order to screen cells at the single cell level, hydrodynamic focusing can be applied by having the buffer flow into the horizontal channel from the two vertical channels at equal flow rates (supported by a second syringe pump). To incorporate electroporation into the device, two platinum electrodes connected to a DC power supply are inserted in the sample and outlet reservoirs to establish an electrical field across the horizontal channel. Electroporation can also be achieved using other types of electrodes and other means known in the art.

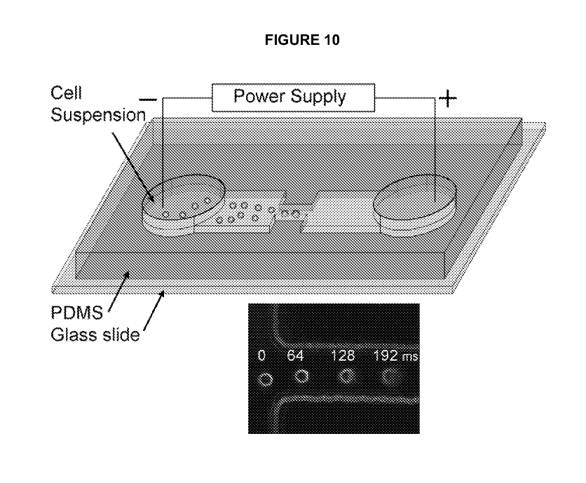

FIG. 10 is another schematic illustration of the electroporative flow cytometry setup. In preferred embodiments, this device can particularly be used for the detection of study of biomechanical properties (features) of cells, and to the detection of protein translocation at the single cell level. Electroporation occurs in the narrow section of the microfluidic channel when cells flow through. Preferably the depth of the channel is about 32 μm and preferably the widths of the narrow and wide section(s) are about 58 μm and about 392 μm, respectively. A constant voltage is established across the channel. A CCD camera can be used to monitor a part of the narrow section including the entry. In one example, the inset image in FIG. 10 shows the cell size change of the same cell (MCF-7) at different time points and at different locations while it is flowing in the channel with the field intensity of 400 V/cm in the narrow section.

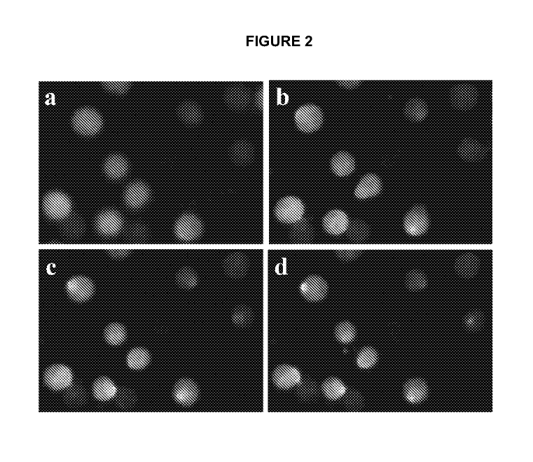

In one aspect of the invention, it was discovered that it is possible to integrate flow-through electroporation with flow cytometry for studying single cells, using so-called electroporative flow cytometry (EFC). Thus, EFC can be applied to detect protein translocation at the single cell level (Wang et al. 2008, Anal. Chem. 80: 1087-1093). In particular, EFC can be applied to detect intracellular translocation of an EGFP-tagged tyrosine kinase, Syk. This is illustrated in FIGS. 2-9.

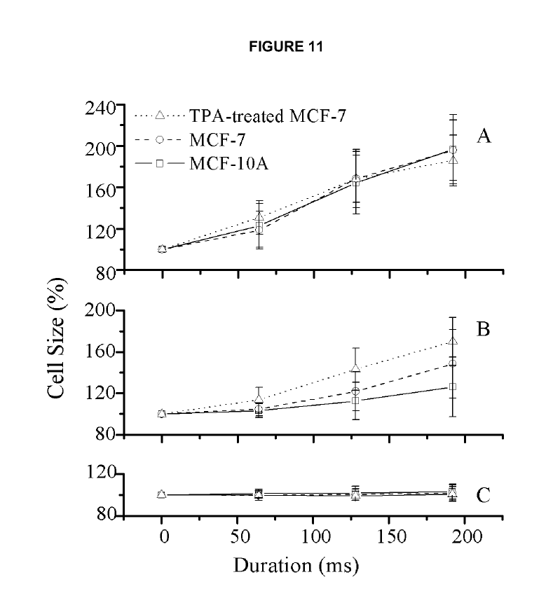

In another aspect of the invention, cell swelling during electroporation can be correlated with the biomechanical properties of the cytoskeleton and/or the deformability of the cell (Bao et al., 2008, Anal. Chem., in press). In some examples, three cell types with different malignant transformation and metastatic potential (MCF-10A, MCF-7 and TPA treated MCF-7) were screened by microfluidic EFC. The swelling of single cells was monitored in real time using a CCD camera with a throughput of about 5 cells/s. Due to their difference in the cytoskeletal mechanics, the cell types were differentiated based on the swelling data collected at the single cell level. Treatment of MCF-7 cells with colchicine, a microtubule polymerization inhibitor, was applied to reveal whether the data obtained were directly relevant to cytoskeletal mechanics and dynamics. The difference in the viability of the cell types after flow-through electroporation was also studied. EFC was validated as an effective and high-throughput tool for studying single cell biomechanics. This is illustrated in FIGS. 11-15.

The devices and methods of the present invention are useful for mechanistic studies of cytoskeletal dynamics and clinical applications such as diagnosis and staging of diseases involving changes in the cell membrane and cytoskeleton in general. EFC can also be applied to heterogeneous cell populations and the quantification of the percentage(s) of malignant/metastatic cells among normal cells can be done by deconvoluting the histogram generated by the mixture, with knowledge of the histograms generated by the individual cell types. Other methods for detecting cell size (e.g. light scattering) can also be used in combination with the devices and methods of this invention. Therefore, this tool can be generally useful for studies of cell biomechanics and cytoskeletal dynamics.

Electroporative flow cytometry (EFC) that combines electroporation with flow cytometry can be used to study deformability of cells at the single cell level. The EFC can be microfluidics-based EFC. The deformability of cells can be correlated to deformability of the cell membrane and/or cytoskeleton. In particular, the degree of cell swelling during or after electroporation treatment is indicative of cell deformability and cytoskeleton/membrane mechanics. More malignant and metastatic cell types exhibit more significant swelling when observed during or after electroporation, due to altered cell deformability. In some cases, about 10% increase in cell size is indicative of increased cell swelling. In other cases, about 20%, 30%, 40%, 50%, 60%, 70%, 80%, 90%, 100%, 150%, or 200% increase in cell size over a corresponding untreated cell is indicative of increased cell swelling. The increase in cell swelling, which can be monitored through the increase in cell size, can serve as the basis for a new label-free detection tool for cancer diagnosis and staging. Furthermore, the cell type with the highest metastatic potential suffers the most cell death due to the flow-through electroporation treatment. Not wanting to be bound by the following theory, this probably occurs due to the most substantial cell swelling irreversibly rupturing the membrane.

The throughput for assaying cells that have altered cytoskeleton and/or cells that are diseased can vary, and in some preferred embodiments it is about 5 cells per second. If desired, cell swelling (as measured by increase in cell size) during electroporation can be recorded in real time.

### EXAMPLES

### Example 1

### Electroporative Flow Cytometry for Detection of Protein Translocation

Microchip fabrication. Microfluidic EFC devices were fabricated based on PDMS using standard soft lithography method described before (Duffy et al., 1998, Anal. Chem. 70: 4974-4984; Wang and Lu, 2006, Anal. Chem. 78: 5158-5164). The microscale patterns were first created using computer-aided design software (FreeHand MX, Macromedia, San Francisco, Calif.) and then printed out on high-resolution (5080 dpi) transparencies. The transparencies were used as photomasks in photolithography on a negative photoresist (SU-8 2010, MicroChem. Corp., Newton, Mass.). The thickness of the photoresist and hence the depth of the channels was around 33 μm (measured by a Sloan Dektak3 ST profilometer). The pattern of channels in the photomask was replicated in SU-8 after exposure and development. The microfluidic channels were molded by casting a layer (approximately 5 mm) of PDMS prepolymer mixture (General Electric Silicones RTV 615, MG chemicals, Toronto, Ontario, Canada) with a mass ratio of A:B=10:1 on the photoresist/silicon wafer master treated with tridecafluoro-1,1,2,2-tetrahydrooctyl-1-trichlorosilane (United Chemical Technologies, Bristol, Pa.). The prepolymer mixture was cured at 85° C. for 2 hours in an oven and then peeled off from the master. Glass slides were cleaned in a basic solution (H₂O: NH₄OH (27%): H₂O₂ (30%)=5:1:1, volumetric ratio) at 75° C. for an hour and then rinsed with DI water and blown dry. The PDMS chip and a glass slide were rendered hydrophilic by oxidizing them using a Tesla coil (Kimble/Kontes, Vineland, N.J.) in atmosphere. The PDMS chip was then immediately brought into contact against the slide after oxidation to form closed channels.

FIG. 1 illustrates a layout of the device fabricated on a microfluidic chip (a), and a schematic illustration of the setup of the microfluidic electroporative flow cytometry apparatus (b). The width varied in the horizontal channel with W₁ of 300 μm and W₂ of 33 μm. The length of the narrow section L₂ was set as either 1 or 2 mm in different experiments. The other sections in the horizontal channel had a length of 2.5 mm for L₁ and 150 μm for L₃. The depth of the microfluidic channels was uniformly 33 μm. The laser detection point was positioned at the center of the horizontal channel after hydrodynamic focusing. In the inset image, the fluorescent trail was left by SykEGFP-DT40-Syk⁻-Lyn⁻ cells when the ratio between the flow rate in one of the two vertical channels and that in the horizontal channel was 3:1.

Cell sample preparation. DT40-Syk⁻-Lyn⁻ and SykEGFP-DT40-Syk⁻-Lyn⁻ cell lines were produced as described before (Ma et al., 2001, J. Immunol. 166: 1507-1516). Both DT40 cell lines were cultured for at least 15 passages in complete medium (RPMI 1640 media supplemented with 10% heat-inactivated fetal calf serum, 1% chicken serum, 50 μM 2-mercaptoethanol, 1 mM sodium pyruvate, 100 IU/ml penicillin G, and 100 μg/ml streptomycin) before the experiment in microfluidic EFC devices. After 48 h subculture, cells were harvested and suspended in serum free electroporation buffer (1 mM MgSO₄, 8 mM Na₂HPO₄, 2 mM KH₂PO₄, and 250 mM sucrose, pH=7.2). One half of the cells (10⁶ cells/mil) were kept unstimulated, while the other half were stimulated with 50 μg/ml goat anti-chicken immunoglobulin M (IGM) antibody (Bethyl Laboratories, Montgomery, Tex.) for 1 h at 20° C.

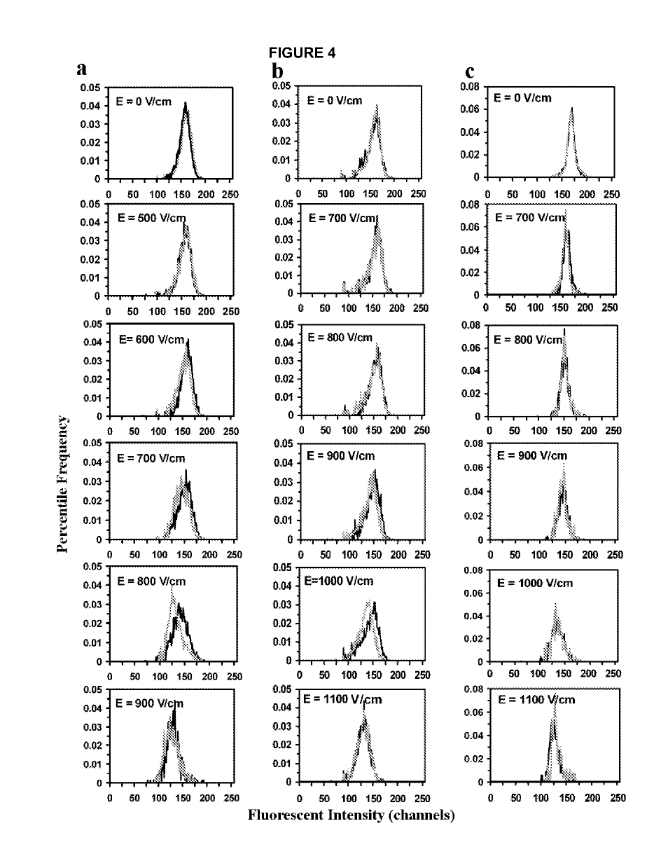

FIG. 4 shows histograms of the fluorescent intensity of DT40 cells detected by the microfluidic EFC under different electric field intensities and durations. SykEGFP-DT40-Syk⁻-Lyn⁻ cells were applied in (a) and (b), while calcein AM stained DT40-Syk⁻-Lyn⁻ cells were used in (c). The black curves were generated by cells stimulated by anti-IgM and the grey curves were obtained from cells without stimulation. The data in (a) and (b) were obtained with different electroporation durations of 120 and 60 ms, respectively. The duration in (c) was 60 ms. The field intensity in the narrow section is indicated for each histogram.

To establish a negative control (FIG. 4c), both stimulated and unstimulated DT40-Syk⁻-Lyn⁻ were labeled with a fluorogenic dye, calcein AM (Invitrogen, Carlsbad, Calif.) after the above procedure for 1 h stimulation or incubation. The labeling was done by incubating the cells with calcein AM at a concentration of 20 ng/ml for 10 min.

All cell samples were centrifuged at 300×g for 10 min before microfluidic EFC experiment with a final cell density of 10⁷ cells/ml in the electroporation buffer described above.

Fluorescence microscopy. The microfluidic device was mounted on an inverted fluorescence microscope (IX-71, Olympus, Melville, N.Y.) with a 40× dry objective (NA=0.60). The epifluorescence excitation was provided by a 100 W mercury lamp, together with brightfield illumination. The excitation and emission from SykEGFP-expressing cells or cells labeled with calcein AM (Molecular Probes, Eugene, Oreg.) were filtered by a fluorescence filter cube (exciter HQ480/40, emitter HQ535/50, and beamsplitter Q5051p, Chroma technology Corp., Rockingham, Vt.).

For the observation of cells after electroporation, cells were transferred from the microchip reservoir to a 96 well plate and then centrifuged for 10 min at 300×g to settle the cells to the bottom before imaging under the microscope. For the real-time observation of kinase translocation in live cells, cell samples were immediately placed on cover slips after adding 50 μg/ml goat anti-IgM antibody in the electroporation buffer (1 mM MgSO₄, 10 mM phosphate buffer, and 250 mM sucrose, pH=7.2) containing the cells.

Microchip operation and signal detection. The setup of the apparatus is shown in FIG. 1. The microfluidic EFC device was mounted on an inverted fluorescence microscope (IX-71, Olympus, Melville, N.Y.) with a 40× dry objective (NA=0.60). Prior to the experiment, the microfluidic channel was flushed with the electroporation buffer for 15 min to condition the channel and remove impurities. The 3 inlets of the channel were connected to a syringe pump (PHD infusion pump, Harvard Apparatus, Holliston, Mass.) through plastic tubing. The volumetric flow rates were set at 1 μl/min for the sample channel inlet and 5 μl/min for each of two side channel inlets. With a cell density of 10⁷ cells/mL, 100-200 cells flowed through the laser detection spot per second. A high voltage power supply (PS350, Stanford Research Systems, Sunnyvale, Calif.) was used to generate a constant direct current (DC) voltage in between the sample and the outlet reservoirs.

An air-cooled 100 mW argon ion laser (Spectra-Physics, Mountain View, Calif.) at 488 nm was applied as the excitation source for laser-induced fluorescence. The laser beam was spectrally filtered by a 10LF10-488 bandpass filter (Newport Corp., Irvine, Calif.) before its intensity was adjusted by neutral density filters (Newport Corp., Irvine, Calif.). The laser was introduced into the microscope through laser port B (Olympus, Melville, N.Y.) and a fluorescence filter cube (505DCLP dichroic beamsplitter, D535/40 emission filter, Chroma Technology Corp., Rockingham, Vt.) before it was finally focused by the objective into the microfluidic channel. The emission light was collected by the same objective and converted into current by a photomultiplier tube (R9220, Hamamatsu, Bridgewater, N.J.) biased at 730 V. The photocurrent was amplified by a low noise current preamplifier (SR570, Standard Research System, Sunnyvale, Calif.) with the cutoff frequency and sensitivity set at 30K Hz and 100 pA/V, respectively. The current was then converted to voltage and input into a PCI data acquisition card (PCI-6254, National Instruments, Austin, Tex.) operated by LabView software (National Instruments, Austin, Tex.). The data were processed by programs written in MATLAB to extract histograms of the fluorescence from a cell population. The data were presented in 4 decades (from 0.001 to 10 V) logarithmic histograms with 256 channels. The voltage signal ranging from 1 mV to 1 V was converted to 4 decade logarithmic voltage scale and then 256 scale channels, due to the small sample size of 2000-3000 cells in each histogram. 100-200 cells per second will go through the laser detection spot.

Conventional flow cytometry. Screening of chicken DT40 cells (stimulated and unstimulated) was conducted using a commercial flow cytometer with 488 nm excitation and FL-1 525 nm BP emission filter (Cytomics™ FC 500 flow cytometer, Beckman Coulter, Inc., Fullerton, Calif.) at the Purdue University Flow Cytometry Laboratories. For each run, around 10000 cells were screened for fluorescence intensity.

Western blot analysis. SykEGFP-DT40-Syk⁻-Lyn⁻ cells (1×10⁶ cells/ml) were treated with or without 50 μg/ml anti-IgM antibody under the conditions described in “cell sample preparation”. The cells were recovered and permeabilized by incubation in a buffer containing 0.1% digitonin, 250 mM sucrose and 1 mM EDTA. The particulate (membrane) fraction was collected by centrifugation at 1000×g, washed once with digitonin free lysis buffer and solubilized in sodium dodecyl sulfate (SDS)-sample buffer to release proteins. The proteins in both soluble (cytosolic) fraction and particulate (membrane) fraction were separated by sodium dodecyl sulfate-polyacrylamide gel electrophoresis (SDS-PAGE), transferred to polyvinylidene difluoride (PVDF) membranes and detected by Western blotting with an anti-Syk antibody (N-19, Santa Cruz Biotechnology, Santa Cruz, Calif.).

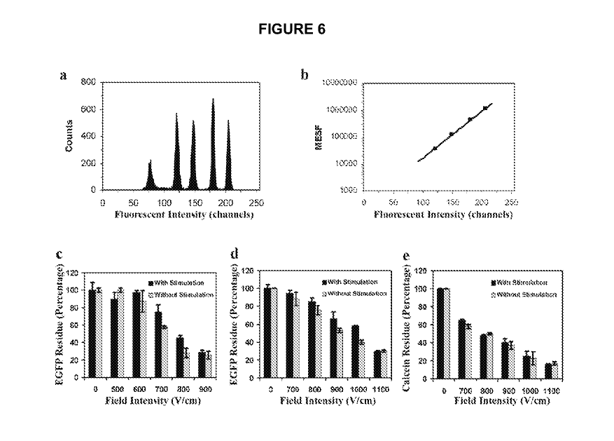

Fluorescence intensity calibration. Molecules of Equivalent Soluble Fluorophores (MESF) units were used to quantify the fluorescence intensity from single cells when microfluidic EFC was used (Wang et at, 2002, J. Res. Natl. Inst. Stand. Technol. 107: 339-353; Martin et al., 2005, Nat. Biotechnol. 23: 1308-1314). A standard curve was first established by screening beads with different predefined fluorescence quantities (high level Quantum FITC MESF Kits, Bangs Laboratories, Inc., Fishers, Ind.) using microfluidic EFC devices. The peak channel numbers of beads was plotted against their known MESF values (FIG. 6b).

FIG. 6 shows graphs illustrating the quantitative analysis of the fluorescence intensity of DT40 cells. (a) Histogram of the fluorescence intensity from a mixture of beads with varying predefined fluorescence intensity levels. The data were generated by the microfluidic EFC system and the population with the lowest intensity is the reference blank. (b) Calibration curve with the MESF values of the beads plotted against the peak channel numbers (the fluorescent intensities) of the beads obtained by the microfluidic system (R²=0.9978, solid square). The mean MESF value of the cells, EGFPₘₑₐₙ in equation (1), is determined by finding the corresponding MESF value for a mean channel value known from the experiments. An assumption was made that the quantity of the fluorescent protein/calcein of the cell population detected by the system without the field (E=0) was 100%. (c) Relative EGFP content in SykEGFP-DT40-Syk⁻-Lyn⁻ cells under different electric field intensities with electroporation duration of 120 ms. (d) Relative EGFP content in SykEGFP-DT40-Syk⁻-Lyn⁻ cells under different electric field intensities with electroporation duration of 60 ms. (e) Relative calcein content in calcein AM stained DT40-Syk⁻-Lyn⁻ under different electric field intensities with a electroporation duration of 60 ms.

The mean percentage of EGFP or calcein of a cell population, P_{EGFP}, (by assuming that SykEGFP-DT40-Syk⁻-Lyn⁻ population without electroporation has a mean percentage of 1 and DT40-Syk⁻-Lyn⁻ population has a mean percentage of 0) was calculated using following formulae (Φ=fluorescence quantum yield, ∈=extinction coefficient, A=absorbance, Peak A_{EGFP}=489 nm, peak A_{Fluorescein}=495 nm, V=average cell volume, EGFPₘₑₐₙ=mean=the population mean MESF value of EGFP fluorescence signal interpolated from the standard curve (FIG. 6b),

[EGFP]=EGFPₘₑₐₙ×B×K/V (1)

where

B=Φ_{EGFP}×∈_{EGFP}×A_{EGFP:488nm/}A_{EGFP:484nm}/(Φ_{Fluorescein}×∈_{Fluorescein}×A_{Fluorescein:488nm/}A_{Fluorescein:490nm})

From the standard curve, K=moles fluorescein/relative fluorescent units. [EGFP]_{control} is the value for DT40-Syk⁻-Lyn⁻ population (without EGFP tagging and without electrical field), [EGFP]_{E=0} is the value for SykEGFP-DT40-Syk⁻-Lyn⁻ population without the electrical field.

By assuming constant B, K and V under different electric field, P_{EGFP} of a cell population can be calculated to be

$${P}_{EGFP} = \frac{( [ EGFP ] - {[ EGFP ]}_{Control} )}{( {[ EGFP ]}_{E = 0} - {[ EGFP ]}_{Control} )} \times 100 \% \qquad ( 2 )$$

The average percentage of calcein in a given cell population was calculated using the same method.

Syk-deficient chicken DT40 cells expressing a fusion protein consisting of Syk coupled to EGFP, SykEGFP, has been shown to respond to anti-IgM antibody stimulation by translocating from cytoplasmic and nuclear compartments to the cross-linked B cell antigen receptor (BCR) at the plasma membrane (Ma et al., 2001, J. Immunol. 166: 1507-1516). In SykEGFP-expressing DT40 cells lacking Syk, treatment with anti-IgM antibody leads to rapid clustering and redistribution of receptor-Syk complexes that form a cap at one pole of the cell. In the absence of Lyn, the receptor-Syk complexes can persist at the inner side of cell membrane without being internalized for more than 1 h at 37° C. Such caps are indicative of the translocation to the plasma membrane.

SykEGFP-expressing chicken DT40 cells lacking both Syk and Lyn were used, to ensure that the localization of Syk at the plasma membrane lasted long enough to finish the tests. The cells were stimulated at room temperature (20° C.) by anti-IgM antibody. Fluorescence images were taken at timed intervals of 0.5 h. FIG. 2 shows that the cells started to show patches and caps of fluorescent clusters at 5 min (FIG. 2a). A large percentage of cells started to form caps at their poles around 30 min after the stimulation. Within 60 min the vast majority of cells had a single cap at one pole. No obvious difference was observed between 60 and 90 min after stimulation with all the caps staying at the membrane. Around 84.2% of cells had capping at around 1 h after anti-IgM antibody stimulation, which is very comparable to 85.4% at 37° C. as previously reported (Ma et al., 2001, J. Immunol. 166: 1507-1516). Based on these results, microfluidic EFC tests were carried out on SykEGFP-DT40-Syk⁻-Lyn⁻ cells at room temperature (20° C.) 1 h after stimulation with anti-IgM when translocation to the plasma membrane was desired.

Conventional flow cytometry is intrinsically insensitive to the subcellular location of the protein of interest. This was confirmed using both a commercial flow cytometer and a microfluidic EFC device with zero voltage.

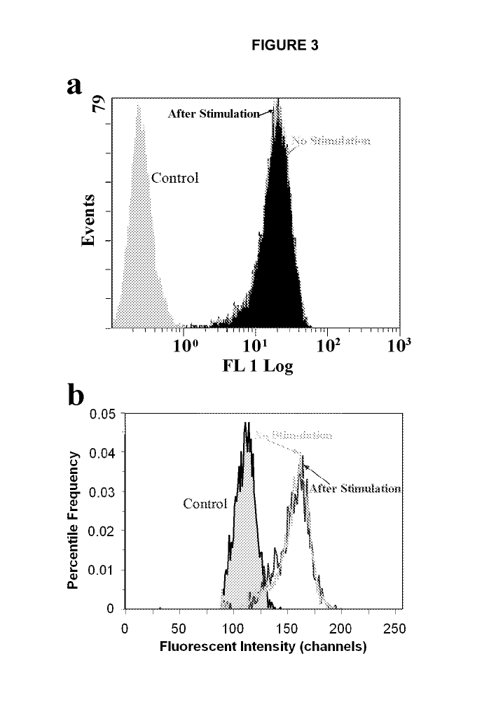

FIG. 3 shows graphs of histograms of the fluorescent intensity of SykEGFP-DT40-Syk⁻-Lyn⁻ cells with and without stimulation by anti-IgM antibody and the DT40-Syk⁻-Lyn⁻ cells (the control). FIG. 3(a) shows histograms obtained by Cytomics FC 500 Flow cytometer (without stimulation; stimulated by anti-IgM; and control, DT40-Syk⁻-Lyn⁻ cells without EGFP labeling). FIG. 3(b) shows analysis of the same samples as in (a) in the microfluidic device (with the voltage at 0). With a commercial flow cytometer the mean fluorescence intensity of the cell population with translocation (after anti-IgM stimulation) was 19.6 compared to 19.2 for the cell population without stimulation, on a 0.1 to 1000 logarithmic relative brightness scale (10,000 cells in each population). To eliminate the effect of different cell sample sizes on the shape of the histograms, the distributions were normalized to have percentile frequency for the y-axis. As shown in FIG. 3, the histograms of the two cell populations overlapped very well. When screening the cells in the microfluidic device of the present invention, the two cell populations (2,000 to approximately 3,000 cells in each population) with and without stimulation also overlapped with the mean channels being 159 and 160, respectively. The results indicate that the translocation cannot be detected with conventional flow cytometry approach.

Microfluidic EFC was used to analyze the cell populations with and without anti-IgM stimulation, under varying voltages across the sample and outlet reservoirs. The field intensity in the narrow section changed from 0 to 1100 V/cm as calculated based on Ohm's law. The field intensity in the wide sections was only about 1/10 of that in the narrow section and electroporation occurred exclusively in the narrow section in all these experiments (Wang and Lu, 2006, Anal. Chem. 78: 5158-5164). The fluorescence intensity from single cells was recorded at the laser focal volume that was close to the exit of the narrow section. The detected signal represented the amount of SykEGFP left in each cell after the electroporation. No interference with the fluorescence signal from the released fluorescent molecules was observed, presumably due to the high flow rate and rapid dilution.

FIG. 4 shows the histograms of the fluorescence intensity from cell populations treated under different conditions and electroporated under different electrical parameters. In each histogram, the y axis shows the percentile frequency of detection and the x axis represents the fluorescence intensity (in channels). In FIG. 4a, histograms of the fluorescence intensity generated by the cell samples stimulated by anti-IgM (blue) and those that were not stimulated (red) are shown, as monitored in the microfluidic EFC device with L₂ of 2 mm. Assuming no effects from the electrical field on the velocity of cells (such effects are generally minor when the velocity of the carrier flow is high), the cells stayed in the narrow section for around 120 ms at this flow rate of 1 μL/min. When the field intensity in the narrow section was increased from 0 to 900 V/cm, the fluorescence intensity of the cell population (stimulated with anti-IgM or not) shifted to the lower end. The translocation did not make any difference in the histogram until the field intensity increased to 600 V/cm. At this field intensity the two histograms did not totally overlap and the stimulated cell population had a slightly higher fluorescence intensity compared to that of the other population without stimulation and translocation. This difference increased further when the field intensity was increased to 700 V/cm and 800 V/cm. At 900 V/cm, the two histograms overlapped again with the mean fluorescence intensity at 128 and 130. When these values were compared to the mean of the control cell population without EGFP-labeling (DT40-Syk⁻-Lyn⁻) at 117, it appeared that the depletion of SykEGFP was not complete at 900 V/cm and 120 ms.

To observe the effects of the duration of time spent in the electroporation field on the release of SykEGFP, a microfluidic EFC device with L₂ of 1 mm was applied. With the flow rates kept the same as in the previous experiment, the electroporation duration before detection was only half of that in the first experiment under the same field intensity. FIG. 4b shows that the two histograms from the cell populations with and without stimulation overlapped very well up to 700 V/cm. Difference between the two histograms started to show up at 800 V/cm and reached a maximum at around 1000 V/cm before the two histograms were not distinguishable again at 1100 V/cm. Compared to the data in FIG. 4a, with the electroporation duration at one half, a higher field intensity was needed to achieve a similar level of differentiation between the stimulated cell population and the unstimulated one. In both FIGS. 4a and b, the difference between the two cell populations was the most pronounced in the medium range of the field intensity. Such a difference was not present without the electric field and it diminished at very high field intensity. To confirm that such differentiation was not caused by the interaction between the plasma membrane and the antibody, the same experiment was repeated with DT40-Syk⁻-Lyn⁻ cells that were not labeled by expression of SykEGFP, but were instead stained with Calcein AM. In live cells, the nonfluorescent calcein AM is converted to green fluorescent calcein, after acetoxymethyl ester hydrolysis by intracellular esterases. As shown in FIG. 4c, there was no significant difference between the cell population with added anti-IgM and the population without the antibody at any field intensity. This confirms that the differentiation was closely related to the translocation of SykEGFP.

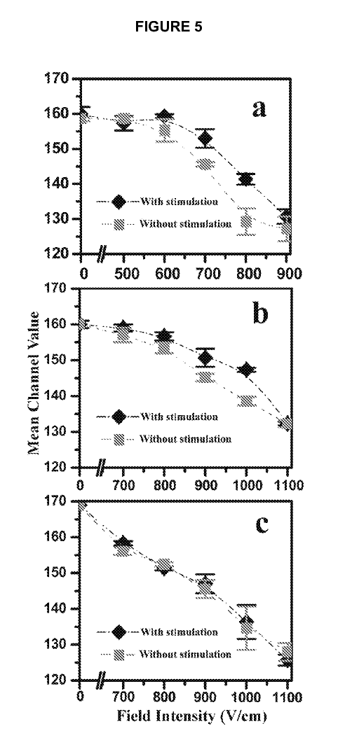

FIG. 5 shows graphs illustrating the variation of the mean fluorescence intensity value of the cell population at different field intensities with and without stimulation by anti-IgM. SykEGFP-DT40-Syk⁻-Lyn⁻ cells were applied in (a) and (b), while calcein AM stained DT40-Syk⁻-Lyn⁻ cells were used in (c). The data in (a) and (b) were obtained with different electroporation durations of 120 and 60 ms, respectively. The duration in (c) was 60 ms. The error bars were generated by carrying out the experiments in triplicate.

FIG. 5 shows the mean fluorescence intensity for each histogram in FIG. 4 plotted against the field intensity for all three experiments. It was found that the optimal field intensity for detecting translocation to the plasma membrane in a cell population was around 800 V/cm with a duration of 120 ms or 1000 V/cm with a duration of 60 ms.

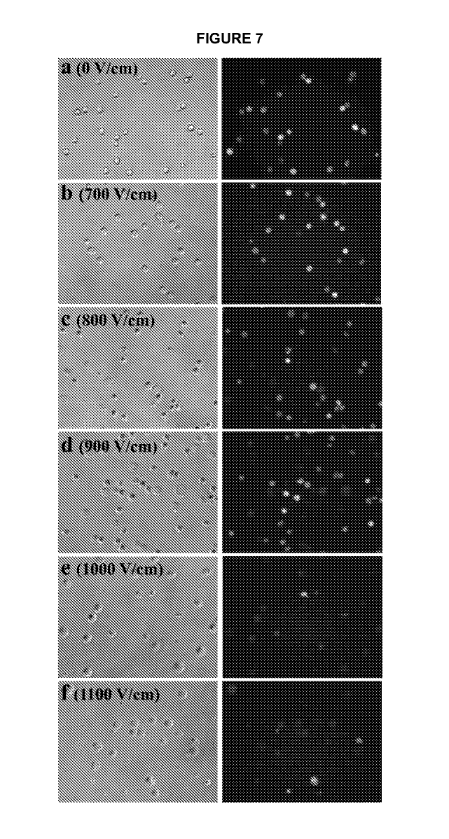

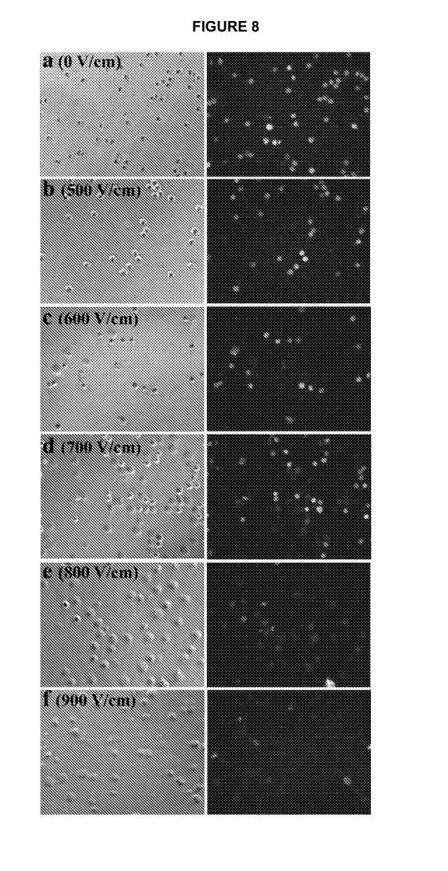

Both phase contrast and fluorescent images of the cells after being processed in the microfluidic EFC device were taken (FIGS. 7, 8). The cell size after the tests became increasingly larger when the field intensity increased, due to the influx of the surrounding solution during electroporation. The residual fluorescence from the cells decreased with increasing field intensity. No significant fragmentation of the plasma membrane was observed. There was some sign of possible membrane fragments only at the highest field intensity and longest duration (e.g., 1100 V/cm and 60 ms, 900 V/cm and 120 ms).

FIG. 7 shows images of SykEGFP-DT40-Syk⁻-Lyn⁻ cells after the microfluidic EFC screening with different field intensities and field duration of 60 ms. The phase contrast images are at the left and the fluorescent images are at the right. All images were taken with the same magnification. The cells were not stimulated.

FIG. 8 shows images of SykEGFP-DT40-Syk⁻-Lyn⁻ cells after the microfluidic EFC screening with different field intensities and field duration of 120 ms. The phase contrast images are at the left and the fluorescent images are at the right. All images were taken with the same magnification. The cells were not stimulated.

Rapid release of intracellular contents during electroporation in similar devices with alternating wide and narrow sections was observed in the inventors' previous work (Wang and Lu, 2006, Biotechnol. Bioeng. 95: 1116-1125; Wang and Lu, 2006, Anal. Chem. 78: 5158-5164; Wang and Lu, 2006, Appl. Phys. Lett. 89: 234102). Such release is due to a combination of outward diffusion and electrophoretic motion of intracellular molecules when the plasma membrane is compromised by the electrical field. The results indicate that the rate of release for an intracellular protein is related to its subcellular location and its interaction with other subcellular molecules and structures, aside from its intrinsic physical properties. It is possible that the binding between Syk and the phosphorylated receptor occurs through a tandem pair of N-terminal SH2 domains was responsible for the higher retention of Syk molecules in the cell population with the translocation to the plasma membrane. Electrical parameters have strong influences on the extent to which a cell population with translocation can be differentiated from one without it.

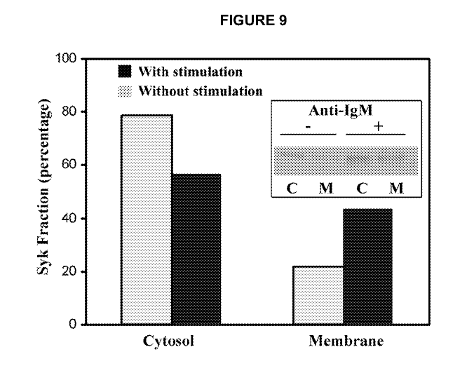

In one aspect, the present invention provides methods for estimating the distribution of a protein kinase at the plasma membrane and in the rest of the cell at the level of the entire population. The accuracy of doing this is largely dependent on the percentage of the kinase undergoing translocation to the plasma membrane. This is in turn related to the number of available binding sites on the plasma membrane. Ideally, if one can completely deplete the EGFP-tagged kinase except for the fraction bound to the plasma membrane, then only the cells with translocation to the plasma membrane would have residual fluorescence after electroporation. However, the results at very high field intensities indicate that the high field intensity and long duration needed for the total depletion of the cytoplasmic and nuclear kinase would possibly dissociate the kinase bound to the plasma membrane or break the plasma membrane into fragments and therefore eliminate the difference between the cells with translocation and those without. In this study the release of the fluorescent protein (SykEGFP) was not totally complete even for the unstimulated cell population without translocation (25.9%-28.6% of the fluorescent protein left in cells with the highest field intensities applied), meaning that a small percentage of the kinase remained in the nucleus or other subcellular compartments at the moment of the detection. Since the stimulus that induces the translocation to the plasma membrane is not likely to cause any significant change in the release of the kinase out of these subcellular compartments, when data from the stimulated population and the unstimulated population are compared, this part of the signal (due to residual kinase) will cancel each other out. The difference between the two populations can still give a very good approximation to indicate the percentage of the kinase translocating to the plasma membrane. By quantifying the fluorescent signal, it was possible to estimate that the average percentage of EGFP-tagged Syk translocating to the plasma membrane in the cell population was around 16.8% (based on data taken with 800 V/cm, 120 ms) and 21.2% (based on data taken with 1000 V/cm, 60 ms) (FIG. 6). These numbers are comparable to 22% obtained using subcellular fractionation/Western blotting under similar stimulation conditions (FIG. 9).

FIG. 9 shows a graph and an image of Western blotting analysis of the SykEGFP fraction in the cytosol and on the membrane with and without stimulation by anti-IgM antibody. The SykEGFP percentages in the cytosol and on the membrane were measured to be 79% and 21%, respectively, without stimulation, while they were 57% and 43% with simulation. Therefore, on average 22% of SykEGFP translocated to the membrane when the cell was stimulated.

By combining electroporation with flow cytometry, microfluidic EFC can be used as a new tool to differentiate cell populations with different activation states based on the subcellular localization of a protein such as a kinase. The methods are applicable to any other protein as well. Thus, microfluidic EFC offers a simple and robust physical tool for detecting protein (e.g. kinase) translocation within the scope of an entire cell population.

In principle, there are no intrinsic limitations that prevent the technology described herein from rivaling flow cytometry in terms of the throughput and stability. The time required for the electroporative release of the intracellular materials from cells does not necessarily put a limitation on the throughput. In principle, the length of the narrow section can be increased to maintain enough duration for the electroporative release of intracellular materials while both the throughput and the velocity of the cells are high. This microfluidics-based technology provides a platform that is well suited for both research and clinical settings and will potentially contribute to drug discovery as well as having utility for both biomarker analysis and patient-stratification.

### Example 2

### Microfluidic Electroporative Flow Cytometry for Studying Single Cell Biomechanics

Cell culture. MCF-7 and MCF-10A cell lines were obtained from American Type Culture Collection (ATCC, Manassas, Va.) and cultured according to recommended protocols. Briefly, MCF-10A cell line was cultured in DMEM F-12 supplemented with Horse Serum (5.6%), EGF (20 ng/ml), Insulin (10 μg/ml), antibiotics (1%) and Hydrocortisone (0.5 μg/ml). The MCF-7 cell line was cultured in DMEM supplemented with FBS (10%), Penicillin/streptomycin (1%) and L-glutamine (2 mM). The TPA treated MCF-7 cell line was generated by treating MCF-7 cells with 100 nM 12-O-tetradecanoylphorbol-13-acetate (TPA) for 18 hr before experiments. In order to detach cells from culture flasks, cells were treated with 0.1% Trypsin/EDTA, washed and resuspended using the electroporation buffer (10 mM Na₂HPO₄, mM NaH₂PO₄, and 250 mM sucrose). For the treatment of MCF-7 cells with colchicine, MCF-7 cells were incubated in the culture medium consisting of 10 μM colchicine (Sigma, St Louis, USA) for 30 min (Rots and Teissie, 1992, Biochim. Biophys. Acta 1111: 45-50). The cell suspension was then centrifuged and the supernatant was discarded. The cells were washed with the electroporation buffer and then resuspended in the electroporation buffer for testing. Safety precautions should be taken; TPA is a potent tumor promoter and colchicine is very toxic. One should be extremely careful when handling the two chemicals.

Microfluidic device fabrication. Microfluidic devices were fabricated on polydimethylsiloxane (PDMS) using standard soft lithography method (Wang and Lu, 2006, Anal. Chem. 78: 5158-5164). Briefly, transparencies with high resolution patterns designed using FreeHand MX (Macromedia, San Francisco, Calif.) were used as photomasks in photolithography. Photoresist SU-8 2025 (MicroChem, Newton, Mass.) was spun on a silicon wafer with a thickness of 32 μm (measured with a Sloan Dektak3 ST profilometer) before it was exposed and developed. PDMS prepolymer mixture consisting of monomer (A) and curing agent (B) (General Electric Silicones RTV 615, MG chemicals, Toronto, Ontario, Canada) with mass ratio of A:B=10:1 was cast on the SU-8/wafer master and heated at 80° C. for 2 hr to generate PDMS chip. The PDMS chip was then peeled off from the master and brought together with a glass slide that was pre-cleaned in a basic solution (H₂O:NH₄OH (27%):H₂O₂ (30%)=5:1:1, volumetric ratio) at 75° C., after both surfaces were oxidized in air using a Tesla coil (Kimble/Kontes, Vineland, N.J.).

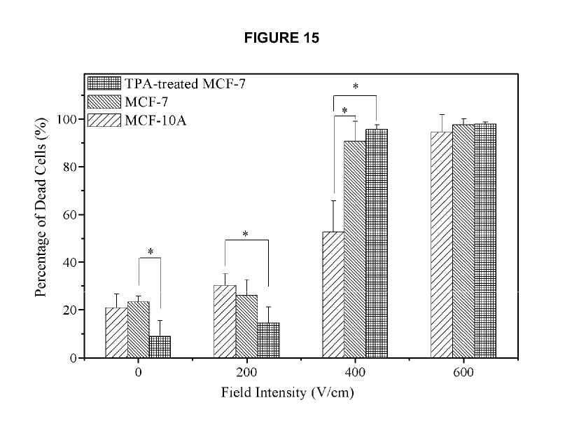

Microchip operation and data collection/analysis. The microfluidic chip (shown in FIG. 10) was mounted on an IX-71 fluorescence microscope (Olympus, Melville, N.Y.) equipped with a 40× dry objective (NA=0.40) and an ORCA-285 CCD camera (Hamamatsu, Bridgewater, N.J.). For EFC tests (data in FIGS. 11, 12, 13, and 14), flow of cells through the microfluidic device was created by maintaining different liquid levels at inlet and outlet reservoirs (as shown in FIG. 10). Given the frame rate of the camera (about 16 Hz), the typical flow velocity of about 0.1-0.25 cm/s provided clear and consecutive images of cells. The cell velocity is influenced by both the flow rate imposed by the syringe pump and the mobility due to the electrical field. However, because the cell size is monitored over time, the results should not be affected by the velocity, assuming mechanical effects such as shear stress can be ignored. For cell viability measurements (data in FIG. 15), a PHD infusion pump (Harvard Apparatus, Holliston, Mass.) was used to create flow with a constant rate which carried cells through the channel. In this case, the duration of electroporation was determined by the flow rate and the dimensions of the microfluidic channel. During all the experiments involving electroporation, a constant voltage was applied across the microfluidic channel using a PS350 power supply (Stanford Research Systems, Sunnyvale, Calif.).

For EFC tests, the images of cells were taken at a frame rate of 16 Hz. The cell size was monitored over time during electroporation by measuring the two-dimensional area (μm²) of individual cells in time-sequenced images using ImageJ software from NIH. As shown in the inset image of FIG. 10, the image of a cell before its coming into the entry of the narrow section of the microfluidic channel was used for calculating the initial cell size (at t=0). The percentile sizes of the same cell in subsequent images were indicated by taking the initial cell size as 1. For cell viability tests, cells were transferred from the receiving reservoir of the device using a pipette to a 96-well plate (each well was pre-loaded with 50 μl culture medium), after they were treated by flow-through electroporation of various field intensities and durations. One hr after the electroporation, propidium iodide (PI) with a concentration of 1 μg/ml was added to stain the cells and then the phase contrast and fluorescent images of the cells were taken for determining the percentile cell death. Dead cells were fluorescently labeled by PI.

As shown in FIG. 10, in EFC experiments, cells flow through a microfluidic channel with a narrow section at the center while constant voltage was established across the channel. Due to the geometry of the channel, the field intensity in the narrow section was roughly 6.8 times higher than that in the wide sections with the wide section width of 392 μm and the narrow section width of 58 μm 32. When the field intensity in the narrow section ranged from 200 to 600 V/cm, the field in the wide sections (<88 V/cm) was substantially lower than the electroporation threshold and did not introduce change in the cell size. A CCD camera was used to record time series of images of cells after they flowed into the entry of the narrow section. In most cases, visible expansion in the cell size was observed immediately after a cell entered the narrow section (as shown in the inset image of FIG. 10).

MCF-10A, MCF-7 and TPA-treated MCF-7 cells, exhibiting different malignancy and metastatic potential, were used as models to validate EFC approach for cancer diagnosis/staging and for biomechanical studies at the single cell level. MCF-10A is a nontumorigenic epithelial cell line derived from benign breast tissue. These cells are immortal, but otherwise normal, noncancerous mammary epithelial cells. MCF-7 is a corresponding line of human breast cancer cells (adenocarcinoma), obtained from the pleural effusion. These cells are nonmotile, nonmetastatic epithelial cancer cells. TPA treated MCF-7 cells were generated by treating MCF-7 cells with 100 nM 12-O-tetradecanoylphorbol-13-acetate (TPA) for 18 hr. The treatment of MCF-7 cells using TPA introduces a dramatic increase (18-fold) in the invasiveness and the metastatic potential of these cells (see Johnson et al., 1999, Exp. Cell Res. 247: 105-113).

FIG. 11 shows graphs illustrating the variation in the cell size during flow-through electroporation for MCF-10A, MCF-7 and TPA-treated MCF-7 cells. The cell size was calculated by measuring the two-dimensional area of the cell at different time points and converting it to percentiles by taking the initial cell size (before entering the narrow section) as 1. Different field intensities in the narrow section (A) 600, (B) 400 and (C) 200 V/cm were applied in the experiments. The duration indicates the time that a cell spent in the narrow section. Each point in these plots was extracted from data of about 50 cells.

As shown in FIG. 11, the swelling of these three cell types with field intensities of 200, 400 and 600 V/cm in the narrow section was examined, and the percentile cell size change during the first 200 ms after the cells flowed into the narrow section was recorded. The data were obtained from single cells and reflected the average and standard deviation of a small cell population. With the electric field strength of 200 V/cm, there was essentially no expansion in the cell size during the 200 ms period for all three cell types, presumably due to the fact that the field intensity was lower than the electroporation threshold. With the increase of the electric field strength to 400 V/cm, all three cell types were observed to swell substantially with TPA-treated MCF-7 swelling the most and MCF-10A the least. At 192 ms after entering the narrow section, the average size of MCF-10A cells increased to 126% of their original size while those of MCF-7 cells and TPA-treated MCF-7 cells increased to 148% and 170%. With further increase of the electric field strength to 600 V/cm, the difference among the cell types diminished despite that each cell line swelled more rapidly compared to at 400 V/cm. The release of intracellular contents accompanies cell swelling during the electroporation process after the membrane is permeabilized.

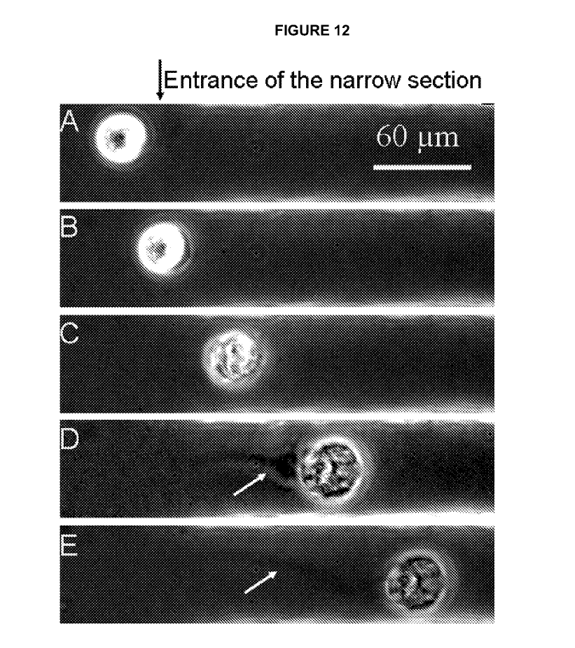

FIG. 12 shows images illustrating the morphological change of the cell during flow-through electroporation and the release of intracellular materials. A TPA-treated MCF-7 cell was monitored at different time points: (A) 0 ms; (B) 64 ms; (C) 128 ms; (D) 192 ms; and (E) 256 ms and various locations in the narrow section. The field intensity in the narrow section was 600 V/cm. The white arrows indicate the visible release of intracellular contents by phase contrast imaging. The position of the entrance of the narrow section is also indicated. FIG. 12 suggests that the release of intracellular materials became pronounced after the first 128 ms at 600 V/cm. This process appears to prevent the continuous expansion in the cell size after the initial period.

The data in FIG. 11 helped to determine a set of electric parameters at which one can differentiate various cell types based on cell swelling during flow-through electroporation. This approach allowed to obtain data on the swelling of each cell in a population, and to put together a histogram about the cell population to indicate the distribution.

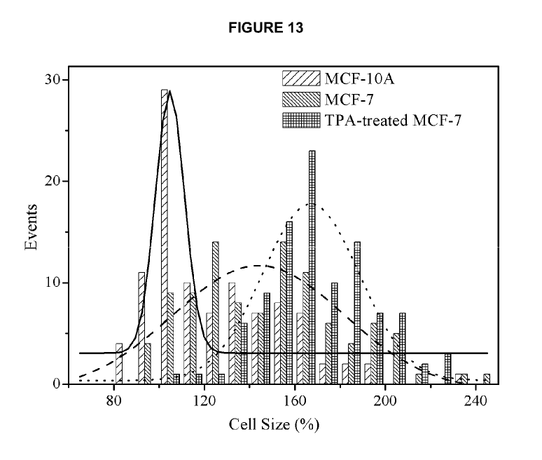

FIG. 13 is a graph showing histograms of the swelling of MCF-10A, MCF-7 and TPA-treated MCF-7 cells under the field intensity of 400 V/cm (in the narrow section) and at the time point of 192 ms. Each histogram includes data from about 100 cells. The curves were added based on the assumption of normal distribution for each population.

As shown in FIG. 13, based on the percentile swelling of a small population of cells with a set of optimized conditions (with 400 V/cm in the narrow section and at 192 ms after electroporation), it was possible to establish the histograms that differentiate the cell types. If these histograms are fit using normal distributions, it can be seen that the average percentile cell size varied from 126% (of the original cell size) for MCF-10A, to 150% for MCF-7 and 170% for TPA-treated MCF-7. The results indicated that the more malignant and metastatic cell line has higher deformability. Similar results on these three cell types were previously obtained using an optical stretcher (Guck et al., 2005; Biophys. J. 88: 3689-3698). Although the average cell size increased substantially during the electroporation for all three cell types, about 15% of MCF-10A cells and 4% of MCF-7 cells had their size decreased (smaller than 100%) as shown in the histograms. In this case, intracellular materials releasing decreased the cell size when the swelling due to the influx was not pronounced for some cells. Such feature would not have been seen if the cells were not examined at the single cell level. The Kolmogorov-Smirnov (K-S) test is applied to compare the two histograms to determine whether the difference is significant (Lampariello, 2000, Cytometry 39: 179-188; Watson, 1992, In: Flow Cytometry Data Analysis: Basic Concepts and Statistics, Cambridge University Press, New York, N.Y.). With 100 data points in each histogram and a confidence level of 99.9%, the critical value Da is 0.2758. The calculations show that the maximum difference (D) between the cumulative distributions is 0.35 for the histograms of MCF-10A and MCF-7 cells, 0.37 for those of MCF-7 and TPA-treated MCF-7 cells, and 0.64 for those of MCF-10A and TPA-treated MCF-7 cells. Because all the maximum differences (D) are larger than the critical value (Da=0.2758), the data yield statistically significant difference between any two cell types.

Colchicine inhibits microtubule polymerization by binding to tubulin. The disruption of microtubules has been shown to cause either cell softening or stiffening (Stamenovic, 2005, J. Biomech. 38: 1728-1732). In the case of MCF-7 cells, the treatment using colchicine causes cell stiffening as shown by EFC data with less swelling during electroporation than the control.

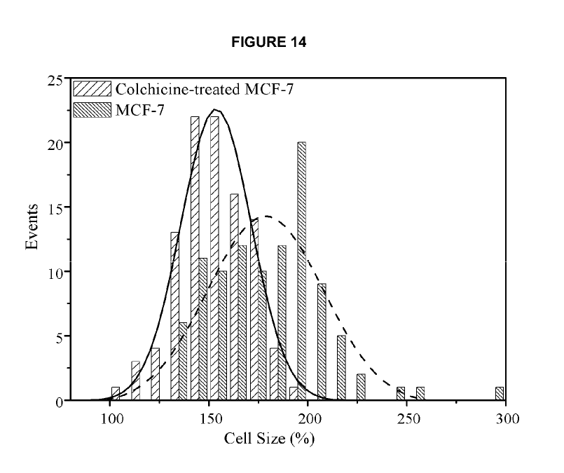

FIG. 14 is a graph showing histograms of the swelling of MCF-7 and colchicine-treated MCF-7 cells under the field intensity of 600 V/cm (in the narrow section) and at the time point of 192 ms. Each histogram includes data from about 100 cells. The curves were added based on the assumption of normal distribution for each population.

FIG. 14 shows that the histograms of MCF-7 cells and colchicine treated MCF-7 cells can be effectively differentiated with selected electrical parameters (duration of 192 ms and an electroporation field intensity of 600 V/cm). K-S test indicates that the difference between the two histograms is significant (D=0.53). The fact that the disruption of microtubules by colchicine has major effects on the results indicates that EFC data have direct links to the cytoskeletal dynamics and mechanics.

The cell viability of the three cell types was examined after flow-through electroporation treatment. The cell types were treated in the device illustrated in FIG. 10 under different electric field strengths with the duration in the narrow section set as 200 ms using a syringe pump which provided a constant flow rate. The cell viability was examined one hr after electroporation using PI staining.

FIG. 15 is a graph showing the viability of MCF-10A, MCF-7 and TPA-treated MCF-7 cells after flow-through electroporation of 200 ms with various field intensities in the narrow section. The percentage of dead cells after the electroporation treatment at 0, 200, 400 and 600 V/cm in the narrow section was determined. Each data point consists of three trials and about 300 cells were examined in each trial. The asterisks indicate statistically significant difference (P<0.05, calculated using student's t test).

As shown in FIG. 15, a low percentage of cells were dead due to exposure to the electroporation buffer (20 mM phosphate and 250 mM sucrose) and possible mechanical damage while flowing through, even without the field. When there was no significant electroporation (0 and 200 V/cm), TPA-treated MCF-7 cells had better survival rate in comparison with the other two cell types due to their metastatic nature. The cell death dramatically increased at 400 V/cm when electroporation started to occur for three cell types. More significantly, at 400 V/cm the cell death for MCF-10A is substantially less than those for MCF-7 and TPA-treated MCF-7. This is likely associated with the fact that more substantial cell swelling was introduced to malignant and metastatic cells. Swelling may lead to cell death by introducing irreversible rupture of the cell membrane. The end result is that normal cells are more resistant to electroporation than malignant and metastatic cells. When the field increased to 600 V/cm, most of the cells were dead for all three cell types. The different cell death rates for cells with different malignancy and metastatic potential under electroporation treatment may provide the basis for targeting of cancerous cells among normal cells.

A throughput of about 5 cells/s was demonstrated using the methods described herein and this throughput is about 300 times higher than that of the fastest single cell technique for cell biomechanics in the literature (Guck et al., 2005, Biophys. J. 88: 3689-3698). In principle, this throughput is limited by the frame rate of the camera used in this study (about 16 frames/s) and can be improved substantially by using a camera with higher speed. Given a sensitive and fast imager, EFC has the potential to yield a throughput comparable to that of conventional flow cytometry (about 10⁴ cells/s).

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art, are within the scope of this invention. All publications, patents, and patent applications cited herein are incorporated by reference in their entirety for all purposes.

What is claimed is:

1. A device comprising
a flow channel having a first section and a second section downstream of the first section and defining a fluid flow path from the first section to the second section,
where the cross-sectional area of the flow channel decreases from the first section to the second section such that upon application of a constant electric field across the flow channel, the electric field intensity in the second section is greater than the electric field intensity in the first section and also greater than the threshold field intensity for electroporation of a cell;
a flow cell comprising means for transporting a stream of particles having cells in suspension such that each cell passes singly through an analysis zone in the second section;
a light source for directing a light beam to intersect the particle stream at the analysis zone, such that only a single cell is exposed to the light beam at one time;
an optical detection means for detecting scattered or fluorescent light pulses from each cell as it passes through the analysis zone; and
electronic means connected to the detection means for converting the light collected by the optical detection means into electrical impulses and analyzing the electrical impulses for the desired information.

2. The device of claim 1 where the constant electric field is generated by constant direct current voltage.

3. The device of claims 1 or 2 where the electric field intensity in the second section is about 5-20 times greater than the electric field intensity in the first section.

4. The device of any one of claims 1 to 3 where the flow, the light source, the optical detection means, and the electronic means comprise fluorescence activated flow cytometry.

5. The device of any one of claims 1 to 4 where the desired information comprises information on protein translocation within the cell.

6. A method of determining protein translocation within a cell by electroporative flow cytometry, the method comprising:
(a) introducing at least one cell comprising a labeled protein into a flow channel of a fluidic device;
(b) subjecting the at least one cell to a constant electric field;
(c) modifying the intensity of the constant electric field,
where the flow channel is configured such that that upon application of the constant electric field through the flow channel, the electric field intensity in one section of the flow channel is greater than the electric field intensity in another section of the flow channel and greater than the threshold field intensity for electroporation for the cell; and
(d) detecting the labeled protein by flow cytometry.

7. The method of claim 6 where modifying the intensity of the constant electric field comprises decreasing the cross-sectional area of the flow channel in the direction of fluid flow.

8. The method of claims 6 or 7 where the flow cytometry comprises fluorescence activated flow cytometry.

9. A method for detecting one or more cells with deformed cytoskeleton, the method comprising:
(a) introducing cells into a flow channel of a fluidic device;
(b) subjecting the cells to a constant electric field;
(c) modifying the intensity of the constant electric field,
where the flow channel is configured such that that upon application of the constant electric field through the flow channel, the electric field intensity in one section of the flow channel is greater than the electric field intensity in another section of the flow channel and greater than the threshold field intensity for electroporation for the cell; and
(d) detecting the cells with increased size by flow cytometry, where the one or more cells with increased size are one or more cells with deformed cytoskeleton.

10. The method of claim 9 where modifying the intensity of the constant electric field comprises decreasing the cross-sectional area of the flow channel in the direction of fluid flow.

11. The method of claims 9 or 10 where detection of the cell size comprises measuring the two-dimensional area of individual cells in time-sequenced images.

12. The method of any one of claims 9-11 where the flow cytometry comprises fluorescence activated flow cytometry.

13. The method any one of claims 9-12 where the cell is a diseased cell.

14. The method of any one of claims 9-13 where the cell is a malignant cell.

15. A method for detecting a diseased cell in a sample, the method comprising:
(a) introducing a cell preparation from the sample into a flow channel of a fluidic device;
(b) subjecting the cell preparation to a constant electric field;
(c) modifying the intensity of the constant electric field,
where the flow channel is configured such that that upon application of the constant electric field through the flow channel, the electric field intensity in one section of the flow channel is greater than the electric field intensity in another section of the flow channel and greater than the threshold field intensity for electroporation for the cell; and
(d) detecting the cell with increased size by flow cytometry, where the cell with increased size over a comparable cell is a diseased cell.

16. The method of claim 15 where modifying the intensity of the constant electric field comprises decreasing the cross-sectional area of the flow channel in the direction of fluid flow.

17. The method of claims 15 or 16 where detection of the cell size comprises measuring the two-dimensional area of individual cells in time-sequenced images.

18. The method of any one of claims 15-17 where the disease is a cytoskeleton-associated disease.

19. The method of any one of claims 15-18 where the cell is malignant.

20. An apparatus, comprising:
(a) means for cell electroporation;
(b) means for moving the electroporated cells, substantially one at a time, in a fluid flow stream;
(c) means for providing an incident beam of illumination directed at the cells in the flow stream;
(d) means for detecting light-related data associated with each moving cell as the cell passes through the beam of illumination; and
(e) means for separating the electroporated cells based on the detected light-related data.

21. The apparatus of claim 20 where the means for electroporation means comprises flow-through electroporation.

22. The apparatus of claims 20 or 21 where the means for detecting light-related data includes means for detecting a plurality of different light signals.

23. The apparatus of any one of claims 20-22 where the means for detecting light-related data includes means for detecting light scattered by the cells and fluorescence emitted from the cells passing through the beam of illumination.

24. The apparatus of claim 23 where the means for detecting light-related data detects the light scatter and fluorescence signals simultaneously.

25. A method, comprising:
(a) subjecting a population of cells to electroporation;
(b) subjecting the population of electroporated cells to flow cytometry; and
(c) detecting flow cytometry-related data associated with a characteristic cellular feature.

26. The method of claim 25 where the cellular feature comprises at least one of protein translocation, cytoskeletal deformation, or cell swelling.

27. The method of claims 25 or 26 where the electroporation is flow-through electroporation.
